(19) United States
(12) Patent Application Publication
(10) Pub. No.: US 2025/0221412 A1
(43) Pub. Date: Jul. 10, 2025

(54) METHODS FOR IRRIGATION USING DELMOPINOL AND DELMOPINOL SALTS

(71) Applicant: You First Services, Inc., Buffalo, NY (US)

(72) Inventor: Satish SHARMA, Buffalo, NY (US)

(21) Appl. No.: 18/850,003

(22) PCT Filed: Mar. 23, 2023
(86) PCT No.: PCT/US2023/064870

§ 371 Date: Sep. 23, 2024

Related U.S. Application Data
(60) Provisional application No. 63/323,050, filed on Mar. 23, 2022.
(60) Provisional application No. 63/322,901, filed on Mar. 23, 2022.

(51) Int. Cl. A01N 43/84; A01P 1/00; A61K 31/5375; A61M 1/00

(52) U.S. Cl. CPC A01N 43/84; A01P 1/00; A61K 31/5375; A61M 1/772; A61M 1/84

(57) ABSTRACT

The present disclosure involves mitigating biofilms through the use of a solution containing delmopinol and/or delmopinol salt. The solution is introduced to the biofilm through various techniques, such as a catheter or flushing a medical device lumen. The biofilm can be located in body cavities, wounds, or on medical devices. The solution is removed by suction or other means and may contain an antibiotic to increase its effectiveness. Agitation of the solution during contact with the biofilm may also be used to increase its efficacy. In some embodiments, the concentration of the solution ranges from 1 μg/l up to a saturated solution, and the contact time can range from 1 second to 5 minutes. The concentrations may be higher or lower, and the contact time may be more or less than these ranges. This method provides an effective and efficient way to mitigate biofilms in various medical fields, including urology, gastroenterology, and wound care.

### CROSS-REFERENCE TO RELATED APPLICATIONS

This application claims priority to U.S. Provisional Application No. 63/322,901, filed on Mar. 23, 2022, now pending, and 63/323,050, filed on Mar. 23, 2022, now pending, the disclosures of which are incorporated herein by reference.

### FIELD OF THE DISCLOSURE

The present disclosure relates generally to the field of medical devices and techniques for mitigating biofilms. More specifically, the disclosure relates to mitigating biofilms in body cavities, wounds, and medical devices using a delmopinol and/or delmopinol salt solution.

### BACKGROUND OF THE DISCLOSURE

Biofilms are complex communities of microorganisms that attach to surfaces and form a protective matrix. Biofilms are a significant problem in medicine, as they can form on medical devices such as catheters, stents, and shunts, as well as within body cavities and open wounds. Biofilms can cause infections, delay wound healing, and decrease the efficacy of antibiotics. Therefore, there is a need for effective methods to mitigate biofilms.

Microbial colonies that form the biofilms use unique methods to attach to surfaces and produce extremely resistant matrices. This is achieved by releasing polysaccharides, lipids, nucleic acids, and proteins to form a protective layer surrounding the bacterium. Infections that follow biofilms lead to devastating diseases like infective endocarditis, pneumonia in cystic fibrosis and repeated urinary tract infection (UTI). There are approximately 1.7 million hospital-acquired infections annually in the United States, incurring an annual economic burden of approximately 11 billion dollars.

As an example, an indwelling Foley catheter is a conduit for bacteria to find entry into the urinary tract and establish colonization of a patient's bladder within three days of their introduction. Bacteria may ascend into the tract via either the external or internal surface of the catheter. Most often microorganisms colonize the external catheter surface by adherence thereby creating a biofilm, usually by the capillary action. Biofilms are complex structures that include bacteria, host cells, and cellular by-products and can develop on human, animal, and plant tissues. Biofilms can also grow on medical devices and implants that have been placed during medical procedures. Biofilm formation subsequent to insertion of the Foley catheters are thought to be a primary mechanism in the development of catheter associated urinary tract infections including some other diseases. Bacteria tend to ascend early after catheter insertion. This suggests a lack of asepsis during initial insertion. Intraluminal bacterial accession tend to be introduced when opening the otherwise closed urinary drainage system. Microbes ascend from the urine collection bag into the bladder via reflux. Any breach or damage to urinary bladder mucosa facilitates biofilm formation on this surface. Biofilms are constituted by cells irreversibly attached to a surface or to each other and embedded in a matrix of extracellular polymeric substances (EPS). Biofilms may form on other devices such as, for example, central venous lines, stents, cerebral shunts, prosthesis, artificial joints etc. and lead to infections.

A biofilm may have a single microorganism or mixtures of many species of bacteria as well as fungi, algae, yeasts, along with dead cells. Only 10% of a biofilm includes the microbial mass with the other 90% contributed by the extracellular matrix, a great deal of which is composed of water. The consistency of biofilms is that of “stiff water” despite the slimy texture they display when viewed macroscopically. An exopolysaccharide production is increased, which could form an exo-polymer slime layer and protect the bacteria against a variety of antimicrobial agents as well as against host attack. Biofilms grow in environments where there is a combination of moisture, nutrients, and a surface. Biofilms are responsible for about 60% of all microbial infections in the human body. Common problems such as urinary tract infection, catheter associated urinary tract infections, ear infections, teeth and gum infections, and contact lenses coatings could all be subsequent to biofilms formed at different anatomical locations.

E. coli is most commonly responsible pathogen for nosocomial infections. Pseudomonas, Enterococcus species, Staphylococcus aureus, coagulase-negative staphylococci, enterobacter species and yeast also are known to cause infection. Proteus and Pseudomonas species are the organisms most commonly associated with biofilm growth on catheters. Risk factors for bacteriuria in patients who are catheterized include longer duration of catheterization, colonization of the drainage bag, diabetes, absence of antibiotics, female gender, renal insufficiency, errors in catheter care, prolonged catheterization in the hospital course, and immuno-compromised or debilitated states. Prolonged antibiotic treatment for 3-14 days on an average as per the studies have shown to reduce the risk of UTI.

Bacteria present in the oral cavity are also present in the genitourinary tract. Although different bacteria may contribute to different components in a biofilm, individually, each one will produce the same matrix at different anatomic locations. Mechanisms of formation of biofilms at different anatomic locations remain the same. Biofilms have a common goal everywhere—to hold bacteria in the biofilms and to lead to colonization and resistant infections.

These infections have a chronic component in common and are very persistent and highly resistant to antimicrobial treatments and host defenses. Acute infections can be removed following a short treatment course of antibiotics. Biofilm-induced infections, however, are usually never completely eliminated and are responsible for recurrent infections. Bacteria in the biofilms could be one thousand times more resistant to antibiotics than the same bacteria grown in liquid medium. It is very difficult to clean biofilms due to the strength with which they adhere to surfaces. This is because the cells produce extracellular polymeric substances (EPS) which form very adhesive gels with water and binds them to the surface. Many biofilms are quite harmful and must be treated or controlled. Despite extensive research into the nature of biofilms, much about the EPS remains a mystery

The approaches for dealing with biofilm-associated problems include:
(1) irrigation of the urinary bladder with a biofilm dissolving compound; (2) catheters, stents, and prosthesis undergoing creative changes; (3) anti-biofilm formulated coatings for catheters, stents, and prosthesis; (4) changes to the materials and architecture of apparatus to help avoid biofilm formation. Additionally, delineating the urinary microbiome of catheter-associated microbes can also provide potential solutions. Despite multiple available options there is still no headway on approaches to effectively solve the problem of biofilm formation on implanted devices and in cavities.

### BRIEF SUMMARY OF THE DISCLOSURE

The present disclosure provides techniques for mitigating biofilms using a delmopinol and/or delmopinol salt solution. The method involves contacting the biofilm with a solution of delmopinol and/or a delmopinol salt. The solution may be introduced to the body cavity, wound, or medical device using a catheter or other suitable means. The solution may be removed using suction or other means. The method may further include agitating the solution during contact with the biofilm.

In some embodiments, the present disclosure provides a method of mitigating a biofilm, the method includes contacting the biofilm with a solution of delmopinol and/or a delmopinol salt (e.g., a sodium delmopinol salt). The delmopinol and/or delmopinol salt solution contacts the biofilm for a pre-determined period of time. For example, the pre-determined period of time may be from 1 second to 20 minutes, inclusive. The method of example 1, wherein the biofilm is in a body cavity. The method may include providing a catheter having a distal end at least partially disposed within the body cavity; and introducing the delmopinol and/or delmopinol salt solution to the body cavity using a catheter. The method may further include removing the delmopinol solution and/or delmopinol salt solution from the body cavity by way of the catheter. For example, the delmopinol and/or delmopinol salt solution is removed using suction.

In some embodiments, contacting the biofilm with a solution includes flushing a lumen of a medical device with the delmopinol or delmopinol salt solution. For example, the medical device may be a catheter, an intravenous line, a stent, a shunt, an irrigation line, and the like. In some embodiments, the medical device does not comprise metal.

In some embodiments, the biofilm is on at least a portion of a prosthesis, and contacting the biofilm with the delmopinol and/or delmopinol salt solution is performed by contacting at least a portion of the prosthesis with the delmopinol and/or delmopinol salt solution. In some embodiments, the biofilm is within at least a portion of an open wound of an individual, and contacting the biofilm with the delmopinol and/or delmopinol salt solution is performed by contacting at least a portion of the open wound with the delmopinol and/or delmopinol salt solution.

In some embodiments, the delmopinol and/or delmopinol salt solution further comprises an antibiotic.

The method may include agitating the delmopinol and/or delmopinol salt solution during contact with the biofilm.

### DESCRIPTION OF THE DRAWINGS

For a fuller understanding of the nature and objects of the disclosure, reference should be made to the following detailed description taken in conjunction with the accompanying drawings.

FIG. 1 is a chart of a method according to an embodiment of the present disclosure.

FIG. 2 is a diagram showing a Foley catheter and a bladder.

### DETAILED DESCRIPTION OF THE DISCLOSURE

Although claimed subject matter will be described in terms of certain embodiments, other embodiments, including embodiments that do not provide all of the benefits and features set forth herein, are also within the scope of this disclosure. Various structural, logical, and process step may be made without departing from the scope of the disclosure.

All ranges provided herein include all values that fall within the ranges to the tenth decimal place, unless indicated otherwise.

As used in this disclosure, the singular forms “a”, “an”, and “the” include plural references and vice versa, unless the context clearly dictates otherwise. Thus, for example, references to “the method” includes one or more methods, and/or steps of the type described herein.

The present disclosure provides methods of mitigating a biofilm using a solution containing delmopinol and/or a delmopinol salt (such as, for example, a sodium salt of delmopinol). The delmopinol and/or delmopinol salt solution may be an aqueous solution. The delmopinol and/or delmopinol salt solution may further comprise a buffer.

The delmopinol and/or the sodium salt of delmopinol solution may have a concentration selected according to the particular application. The concentration may be selected to be a therapeutically effective concentration. In some embodiments, the concentration is between 1 μg/liter up to a saturated solution, inclusive. For example, in various non-limiting embodiments, the concentration of delmopinol and/or delmopinol salt may be between 1 mg/l and 5 g/l, 5 mg/l and 2 g/l, or 10 mg/l and 1 g/l, in each case inclusive. For example, the concentration may be 1, 2, 3, 4, 5, 10, 15, 20, 25, 30, 35, 40, 45, 50, 75, or 100 mg/l. The concentration may be higher or lower than these exemplary values and ranges.

Delmopinol has the following structure:

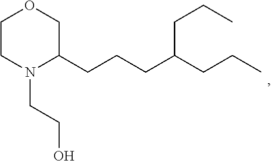 [SMILES: CCCC(CCC)CCCC1COCCN1CCO]

and the sodium salt of delmopinol has the following structure:

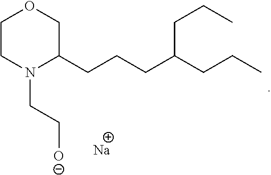 [SMILES: CCCC(CCC)CCCC1COCCN1CC[O-] ; [Na+]]

Delmopinol and its sodium salt may be made by the following synthetic route:

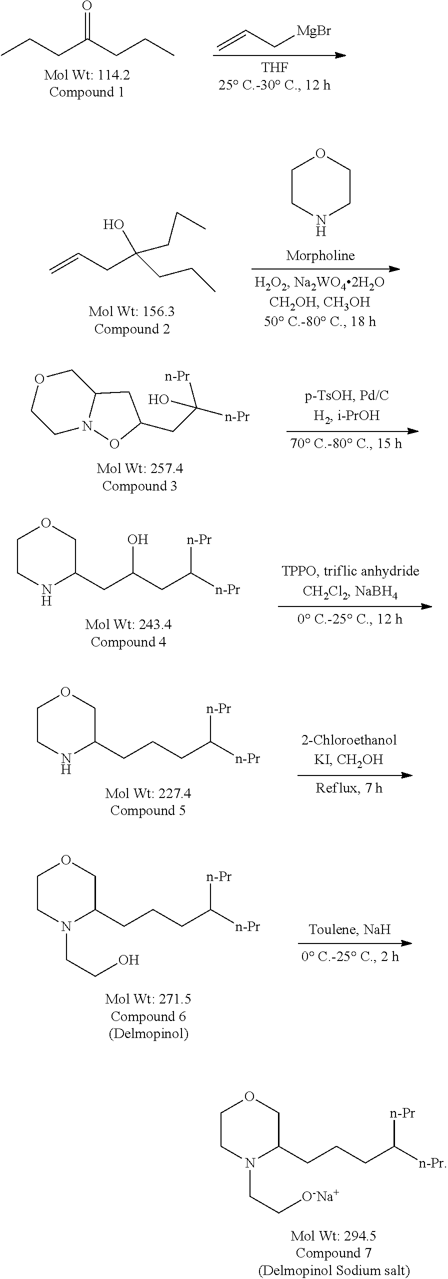

Without intending to be bound by any particular theory, it is considered that delmopinol and its salts (e.g., sodium salt of delmopinol) can remove biofilms and inhibit formation of biofilms.

In some embodiments, the delmopinol and/or delmopinol salt solution may further include an antibiotic. Non-limiting examples of suitable antibiotics include penicillins, cephalosporins, tetracyclines, aminoglycosides, macrolides, macrolides, sulfonamides, quinolones, lincosamides, glycopeptides, and the like. Additional examples of antibiotics include, but are not limited to, phenoxymethylpenicillin, dicloxacillin, ampicillin, facillin, oxacillin, penicillin V, penicillin G, flucloxacilline, amoxicillin, cefaclor, cefadroxil, cephalexin, cefazolin, cefuroxime, cefixime, cefoxitin, ceftriaxone, tetracycline, doxycycline, minocycline, sarecycline, lymecycline, gentamicin, tobramycin, amikacin, planomycin, streptomycin, neomycin, paromomycin, erythromycin, azithromycin, clarithromycin, clindamycin, fidaxomicin, roxithromycin, trimethoprim, co-trimoxazole, metronidazole, tinidazole, ciprofloxacin, levofloxacin, norfloxacin, ofloxacin, moxifloxacin, nitrofurantoin, sulfamethoxazole, sulfasalazine, sulfacetamide, sulfadiazine silver, vancomycin, dalbavancin, oritavancin, telavancin, and the like. Other antibiotics are known in the art and are contemplated for use with the delmopinol and/or delmopinol salt solution.

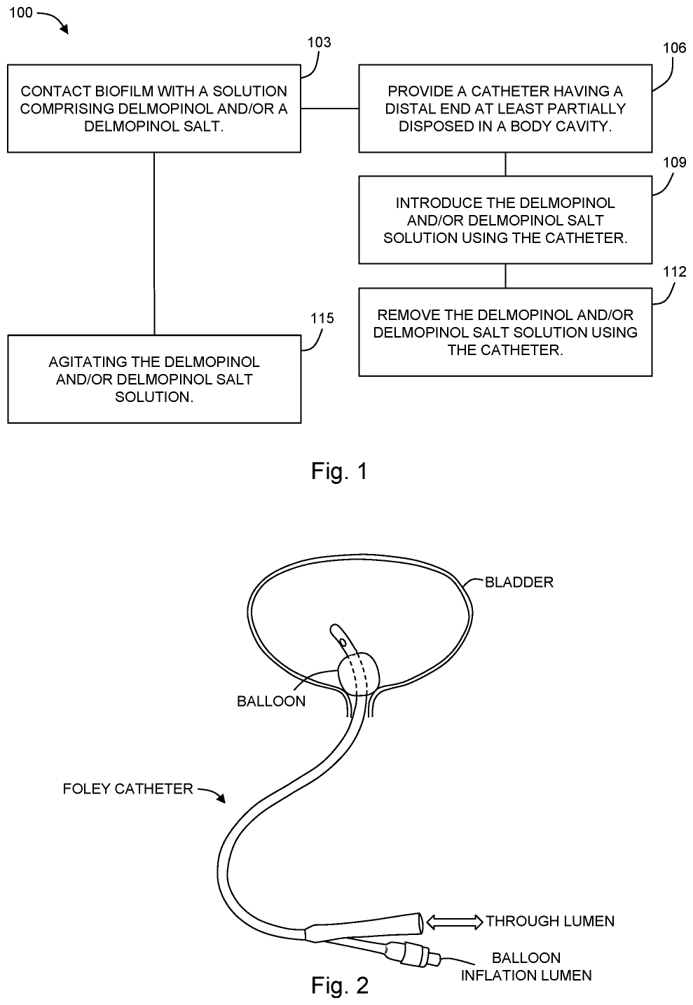

With reference to FIG. 1, the present disclosure may be embodied as a method 100 for mitigating a biofilm. The method 100 includes contacting 103 the biofilm with a delmopinol and/or delmopinol salt solution, such as, for example, the delmopinol and/or delmopinol salt solutions described herein (e.g., a metal salt, such as, for example, a sodium salt or calcium salt of delmopinol). The delmopinol and/or delmopinol salt solution may contact the biofilm for a pre-determined period of time, where the pre-determined period of time is selected to be therapeutically effective (i.e., able to at least partially mitigate the biofilm). For example, the pre-determined period of time may range from 1 second to 20 minutes, inclusive, for example, 2, 3, 4, 5, 10, 15, 20, 25, 30, 45, or 60 seconds. The pre-determined period of time may be longer or shorter.

In some embodiments, the biofilm is in a body cavity. The method 100 may further comprise providing 106 a catheter having a distal end at least partially disposed with the body cavity. The delmopinol and/or delmopinol salt solution may then be introduced 109 to the body cavity by way of the catheter. For example, a syringe may be used to push delmopinol and/or delmopinol salt solution through the catheter into the body cavity. In a particular, non-limiting example (shown in FIG. 2), the biofilm may be in a bladder, and the delmopinol and/or delmopinol solution may be introduced by way of a Foley catheter. In some embodiments, the method 100 further includes removing 112 the delmopinol and/or delmopinol salt solution from the body cavity using the catheter. For example, suction (e.g., syringe, etc.) may be used to remove the solution. In other embodiments, the solution may drain via gravity, bodily action, or otherwise.

In some embodiments, contacting 103 the biofilm with the delmopinol and/or delmopinol salt solution is performed by flushing a lumen of a medical device. For example, the medical device may be a central line, such as, for example, a central venous line, and the line may be flushed using delmopinol and/or delmopinol salt solution. In another example, the medical device may be a dental chair line (e.g., used to provide irrigation and/or other fluids to a dental chairside), and the method includes contacting the biofilm by flushing the dental chair line using delmopinol and/or delmopinol salt solution. Other medical devices include, but are not limited to an intravenous line, a stent, a shunt, and an irrigation line. In some embodiments, the medical device does not comprise metal (i.e., at least the portion of the medical device having a lumen does not include metal).

In some embodiments, the biofilm is within at least a portion of an open wound of an individual. In such cases, the delmopinol and/or delmopinol salt solution is contacted with the biofilm by contacting at least a portion of the open wound with the solution. In some embodiments, the biofilm is on at least a portion of a prosthesis. In such cases, contacting the biofilm with the delmopinol and/or delmopinol salt solution is performed by contacting at least a portion of the prosthesis with the delmopinol and/or delmopinol salt solution.

In some embodiments, the method 100 includes agitating 115 the delmopinol and/or delmopinol salt solution during contact with the biofilm. For example, in an example where a syringe is used to introduce the solution into a catheter, the syringe may be used to move the solution into and out of the lumen to agitate.

The following Examples provide various embodiments of the present disclosure.

Example 1. A method of mitigating a biofilm, the method comprising contacting the biofilm with a solution comprising delmopinol and/or a delmopinol salt (e.g., a sodium delmopinol salt).

Example 2. The method of example 1, wherein the delmopinol and/or delmopinol salt solution contacts the biofilm for a pre-determined period of time.

Example 3. The method of example 2, wherein the pre-determined period of time is from 1 second to 20 minutes, inclusive.

Example 4. The method of example 1, wherein the biofilm is in a body cavity, and further comprising: providing a catheter having a distal end at least partially disposed within the body cavity; and introducing the delmopinol and/or delmopinol salt solution to the body cavity using a catheter.

Example 5. The method of example 4, further comprising removing the delmopinol solution and/or delmopinol salt solution from the body cavity by way of the catheter.

Example 6. The method of example 5, wherein the delmopinol and/or delmopinol salt solution is removed using suction.

Example 7. The method of example 1, wherein contacting the biofilm with a solution comprises flushing a lumen of a medical device with the delmopinol or delmopinol salt solution.

Example 8. The method of example 7, wherein the medical device is a catheter, an intravenous line, a stent, a shunt, an irrigation line, and the like.

Example 9. The method of example 8, wherein the medical device does not comprise metal.

Example 10. The method of example 1, wherein the biofilm is on at least a portion of a prosthesis, and contacting the biofilm with the delmopinol and/or delmopinol salt solution is performed by contacting at least a portion of the prosthesis with the delmopinol and/or delmopinol salt solution.

Example 11. The method of example 1, wherein the biofilm is within at least a portion of an open wound of an individual, and contacting the biofilm with the delmopinol and/or delmopinol salt solution is performed by contacting at least a portion of the open wound with the delmopinol and/or delmopinol salt solution.

Example 12. The method according to any one of examples 4-11, wherein the delmopinol and/or delmopinol salt solution contacts the biofilm for a pre-determined period of time.

Example 13. The method of example 12, wherein the pre-determined period of time is from 1 second to 5 minutes, inclusive.

Example 14. The method according to any one of examples 1-13, wherein the concentration of the delmopinol and/or delmopinol salt solution is from 1 μg/l up to a saturated solution, 1 mg/l and 5 g/l, 5 mg/l and 2 g/l, or 10 mg/l and 1 g/l, inclusive.

Example 15. The method according to any one of examples 1-14, wherein the delmopinol and/or delmopinol salt solution further comprises an antibiotic.

Example 16. The method according to any one of examples 1-15, further comprising agitating the delmopinol and/or delmopinol salt solution during contact with the biofilm.

Although the present disclosure has been described with respect to one or more particular embodiments and/or examples, it will be understood that other embodiments and/or examples of the present disclosure may be made without departing from the scope of the present disclosure.

We claim:

1. A method of mitigating a biofilm, the method comprising:
contacting the biofilm with a solution comprising delmopinol and/or a delmopinol salt.

2. The method of claim 1, wherein the delmopinol and/or delmopinol salt solution contacts the biofilm for a pre-determined period of time.

3. The method of claim 2, wherein the pre-determined period of time is from 1 second to 20 minutes, inclusive.

4. The method of claim 1, wherein the biofilm is in a body cavity, and further comprising:
providing a catheter having a distal end at least partially disposed within the body cavity; and
introducing the delmopinol and/or delmopinol salt solution to the body cavity using a catheter.

5. The method of claim 4, further comprising removing the delmopinol solution and/or delmopinol salt solution from the body cavity by way of the catheter.

6. The method of claim 5, wherein the delmopinol and/or delmopinol salt solution is removed using suction.

7. The method of claim 1, wherein contacting the biofilm with a solution comprises flushing a lumen of a medical device with the delmopinol or delmopinol salt solution.

8. The method of claim 7, wherein the medical device is a catheter, an intravenous line, a stent, a shunt, an irrigation line, and the like.

9. The method of claim 8, wherein the medical device does not comprise metal.

10. The method of claim 1, wherein the biofilm is on at least a portion of a prosthesis, and contacting the biofilm with the delmopinol and/or delmopinol salt solution is performed by contacting at least a portion of the prosthesis with the delmopinol and/or delmopinol salt solution.

11. The method of claim 1, wherein the biofilm is within at least a portion of an open wound of an individual, and contacting the biofilm with the delmopinol and/or delmopinol salt solution is performed by contacting at least a portion of the open wound with the delmopinol and/or delmopinol salt solution.

12. The method according to any one of claims 4-11, wherein the delmopinol and/or delmopinol salt solution contacts the biofilm for a pre-determined period of time.

13. The method of claim 12, wherein the pre-determined period of time is from 1 second to 5 minutes, inclusive.

14. The method according to any one of claims 1-13, wherein the concentration of the delmopinol and/or delmopinol salt solution is from 1 μg/l up to a saturated solution, 1 mg/l and 5 g/l, 5 mg/l and 2 g/l, or 10 mg/l and 1 g/l, inclusive.

15. The method according to any one of claims 1-14, wherein the delmopinol and/or delmopinol salt solution further comprises an antibiotic.

16. The method according to any one of claims 1-15, further comprising agitating the delmopinol and/or delmopinol salt solution during contact with the biofilm.
